(19) United States
(12) Patent Application Publication
(10) Pub. No.: US 2020/0190509 A1
(43) Pub. Date: Jun. 18, 2020

(54) METHODS FOR IDENTIFYING ACTIVATORS AND SUPPRESSORS

(71) Applicant: KATHOLIEKE UNIVERSITEIT LEUVEN, Leuven (BE)

(72) Inventors: Cathérine VERFAILLIE, Leuven (BE); Béla SCHMIDT, Evere (BE)

(73) Assignee: Katholieke Universiteit Leuven, Leuven (BE)

(21) Appl. No.: 16/619,697

(22) PCT Filed: Jun. 6, 2018
(86) PCT No.: PCT/EP2018/064858

§ 371 Date: Dec. 5, 2019

(30) Foreign Application Priority Data
Jun. 6, 2017 (GB) 1708998.8

(51) Int. Cl. C12N 15/10; C12N 15/11; C12N 9/22; C12N 15/85

(52) U.S. Cl. CPC C12N 15/1086; C12N 15/11; C12N 9/22; C12N 2320/12; C12N 2310/20; C12N 2800/80; C12N 15/85

(57) ABSTRACT

The present invention relates to screening methods for identifying suppressors or activators of a pathway.

### FIELD OF THE INVENTION

The present invention relates to methods for identifying genes in a pathway. The present invention further relates to mutagenesis based on RNA-guided nucleases (such as CRISPR/Cas9), and screening methods based on reporter genes with selectable markers.

### BACKGROUND OF THE INVENTION

The industry standard for high-throughput screening now is arrayed screening. Arrayed screens allow for comprehensive molecular readouts such as transcriptome profiling, but such readouts limit throughput.

Pooled screens require simple readouts including cell proliferation and sortable marker proteins, which restricts the problems that can be studied.

For genome-wide loss-of function screen in mammalian cells, the current standard is RNAi, but it has many problems: the scope is limited to mature transcripts, depletion at the protein level is partial (usually in the range of 70%), and there are confounding off-target effects.

CRISPR/Cas9 for generating gene knockouts can solve the problem of partial knock-down, and using several gRNAs per gene offers a good way of controlling for off-target effects.

Pooled CRISPR screens work well for mechanisms that affect cell survival and proliferation, and they can be extended to measure the activity of individual genes (e.g., by using engineered reporter cell lines). sgRNAs are usually delivered using lentiviral or retroviral vectors, which ensures integration into the genome of the target cell at controlled copy number. Alternatively, other viral vectors such as AAV may also be used [see Kennedy et al. (2015) Virology 479-480, 213-220]. Several publications have shown the feasibility of pooled CRISPR/Cas9 screens (Gilber et al. (2014) Cell 159, 647-661; Shalem et al. (2015) Nat Rev Genet 16, 299-311; Konermann et al. (2015) Nature 517, 583-588; Parnas et al. (2015) Cell 162, 675-686).

The pooled CRISPR/Cas9 screens described in the literature rely on either a special phenotype present in the cell line used for screening (resistance to certain drugs conferred by mutant protein present in the cell line) or a FACS-sortable phenotype. This restricts the questions that can be answered with the screen to questions where special mutants are available or, in case of FACS sorting, to relatively frequent events (so the number of cells that needed to be screened is not impractically high to find cells with the desired phenotype).

Several publications (Adamson et al. (2016) Cell 167, 1867-1882; Datlinger et al. (2017) Nat Methods 14, 297-301; Dixit et al. (2016) Cell 167, 1853-1866; Norman et al. 2016, Parnas et al. 2016; Jaitin et al. (2016) Cell 167, 1883-1896, Rendeiro et al. 2017) have managed to use single-cell RNA sequencing as the readout of pooled CRISPR/Cas9 screens, but not at the full genome level.

### SUMMARY OF THE INVENTION

The present invention is applicable with pooled screens for all pathways that end with upregulation of a reporter gene (preferably with a wide dynamic range). By the use of positive and negative selection markers in combination with triggering the pathway, the cell population is enriched for either activators or suppressors of the pathway.

The relative abundance of the gene targeting agents (gRNAs) is then compared with the baseline abundance. The results of the screen can be analysed using the bioinformatics tools established for analysing RNAi screens (Miles et al. (2016) Febs J. 283, 3170-3180)

The method is using positive selection (enrichment of relevant cells containing a knock-out of an activator or suppressor of a gene in a pathway) to identify both activators and suppressors of the pathway in question.

The invention is summarised by the following statements:

1. A method for pooled screening using RNA-guided nucleases for identifying genes in a pathway of interest comprising the steps of:
providing a cell line with an CRISPR/Cas9 expression cassette,
transfecting said cell line with a reporter construct of a reporter gene in a pathway of interest wherein the reporter construct comprises at least the regulatory elements of said reporter gene, operably linked to a positive and negative selection marker, whereby a modified expression of the reporter gene leads to a modified expression of the positive and negative selection marker,
introducing in said cell line with said reporter construct a pooled library of CRISPR/Cas9 guide RNA,
cultivating said population of cells,
inducing Cas9 activity, thereby introducing a modification in the genome of cells which comprise a guide RNA,
activate the pathway of interest and contact part of the cells with a positive selection agent and part of the cells with a negative selection agent

wherein cells which survive treatment of the positive selection agent are indicative of being cells wherein a suppressor of the pathway is inactivated, and wherein cells which survive treatment with the negative selection agent are indicative of being cells wherein an activator of the pathway is inactivated.
identifying within the cells with increased viability, the guide RNA within the cells, where the gene that has been targeted is identified as a gene involved in the pathway.

2. The method according to statement 1, wherein the reporter gene is a gene which is constitutively expressed, allowing the measurement of a downregulation or an upregulation of the activity of the reporter gene,

or wherein, during control and experimental conditions, the reporter gene is activated to a certain level, allowing the measurement of a downregulation or an further upregulation of the activity of the reporter gene.

3. The method according to statement 1 or 2, wherein the CRISPR/Cas9 expression cassette is an inducible CRISPR/Cas9 expression cassette.

4. The method according to any one of statements 1 to 3, whereby an increased expression of the reporter gene leads to an increased expression of the positive and negative selection marker, or

whereby a decreased expression of the reporter gene leads to a decreased expression of the positive and negative selection marker.

5. The method according to any one of statements 1 to 4, wherein the reporter construct comprises the regulatory elements of said reporter gene and the coding sequence of the reporter gene, and wherein the reporter gene is operably linked to a positive and negative selection marker.

6. The method according to any one of statements 1 to 5, wherein the positive marker is gene which expression converts a toxic positive selection agent into a non-toxic compound.

7. The method according to statement 6, wherein the positive marker is puromycin N-acetyl-transferase (PURO) and the positive selection agent is puromycin.

8. The method according to any one of statements 1 to 5, wherein the negative marker is gene which expression converts a non-toxic negative selection agent into a toxic compound.

9. The method according to statement 8, wherein the negative marker is thymidine kinase and the negative selection agent is FiAU.

10. The method according to any one of statements 1 to 9, wherein the cells transfected with the expression cassette are stem cells.

11. The method according to statement 10 wherein prior to the induction of Cas9 said stem cells are differentiated into a desired cell type.

12. The method according to statement 10 or 11, wherein the stem cells are iPSC cells.

13. The method according to any one of statements 10 to 12, wherein the stem cells are differentiated into stellate cells.

14. The method according to any one of statements 1 to 13, wherein the RNA library is a barcoded library and wherein the identity of the gene involved in the pathway of interest is identified by the barcode corresponding to the guide RNA.

15. The method according to any one of statements 1 to 14, further comprising the step of determining the expression of the protein, the level of mRNA, or determining the genomic sequence of the gene.

### FIGURE LEGENDS

FIG. 1 illustrates the fate of cells in which a reporter gene is tagged with a puromycin resistance gene/thymidine cassette when such cells are treated with respectively puromycin and FiAU ((1-(2′-deoxy-2′-fluoro-1-(3-D-arabinofuranosyl)-5-iodouracil) when the reporter is not activated (top part) or activated (bottom part). When the reporter is inactive puromycin will kill the cells, the harmless compound FiAU is not converted into its toxic phosphorylated version.

When the reporter is active the puromycin resistance gene (Puro) protects the cells against puromycin. FiAU is converted into its toxic phosphorylated version by the activity of thymidine kinase (TK).

FIG. 2 illustrates the fate of above mentioned cells upon treatment with puromycin and FiAU for control cells (non-edited), and cells with a disrupted activator or suppressor of the pathway.

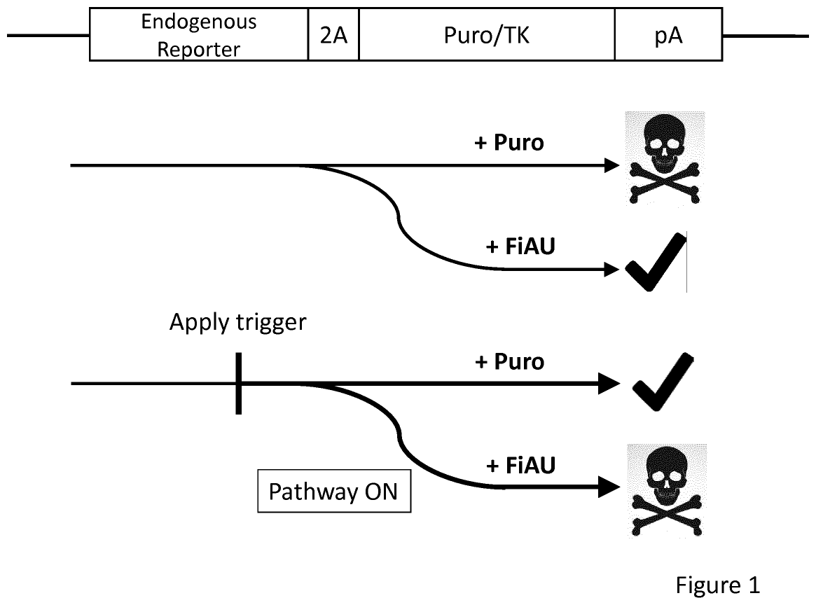

The control conditions of the experiment use already some extent of activation of the pathway to ensure expression of the positive selection marker gene (PUR) such that cell growth is achieved in the presence of the positive selection agent puromycin.

Left bars show the expression level of Pur/TK when the pathway is activated. Right bars show the expression level of Pur/TK when the pathway is not-activated. The expression level in normal activated cells is set at 100. The expression level in normal non-activated cells is close to 0 (some leaky expression may occur).

Expression levels are lower in cells wherein activators are knocked-out. (also in the OFF condition the leaky expression will be even lower).

Expression levels are higher in cells wherein activators are knocked-out. (also in the OFF condition the leaky expression will be slightly higher).

The following events are represented below the figure.

1 Non-edited cells

1a. pathway off: Puromycin has a toxic effect (cells die); FiAU has no toxic effect (cells survive)

1b pathway on: Puro expression protects cells against Puromycin; TK expression converts FiAU and kill cells.

2. Activator KO cells

2a. pathway off: Puromycin has a toxic effect (cells die); FiAU has no toxic effect (cells survive)

2b. pathway on: The reporter is less activated, Less Puro is expressed making the cells less resistant to Puromycin; Less TK is expressed making the cells more resistant to FiAU.

3. suppressor KO cells

3a. pathway off: Puromycin has a toxic effect (cells die); Fiau has no toxic effect (cells survive).

3b. pathway on: The reporter is more active. More Puro is expressed making the cells more resistant to Puromycin; more TK is expressed making the cells less to FiAU.

FIG. 3 illustrates which conditions allow selective enrichment of cells that are knocked out of an activator or a suppressor of the pathway of interest.

FIG. 4 gives an overview the fate of cells upon treatment with puromycin and FiAU, in control settings.

FIG. 5 gives an overview the fate of cells upon treatment with puromycin and FiAU, in populations wherein activators or suppressors are knocked out.

### DETAILED DESCRIPTION

The main components of the system are:

1. Reporter gene with a wide dynamic range and a (preferably titratable and physiological) trigger.

2. Cell line or any other organism with (preferably) inducible expression of a genome perturbing agent, and a reporter gene tagged with a positive and negative selection marker. The present invention is typically performed on human cell lines (including stem cells and differentiated cells), but can be equally applied on other mammalian, or eukaryote cells, and even on whole organisms, as long as the required gene engineering can be performed.

The genome perturbing agent can be any agent changing the expression pattern, expression level, or function of any gene in the genome.

In specific embodiments, the creation of the cell lines with reporter construct, and gene perturbating system is introduced in a stem cells. The selection of suppressors and activators of a pathway by triggering the pathway and selection cells with increased viability is performed on differentiated cells.

Typically the genome perturbing agent is the CRISPR/Cas9 system. Other RNA-guided nucleases, or other mutagenesis systems, such as CRISPR/dCas-interference/activation (e.g. combinations of a dead Cas with a KRAB domain to recruit transcriptional repressors that interfere reversibly and inducibly with gene expression at levels of ±90% (CRISPRi); or with a sequence such as VP16, VPR or Sun-Tag to activators to endogenous genes to enable inducible and reversible and activation (CRISPRa)), transposon based mutagenesis, or even chemical mutagenesis or irradiation can be used. Systems wherein an unknown mutation in the genome can be easily traced are preferred [Kampmann M. (2017) Trends Mol Med. 2, 483-485].

In the context of the present invention a “positive selection marker” is a gene product that allows the survival of the cells during treatment with an agent that kills cells not expressing the positive selection marker.

In the context of the present invention, “negative selection marker” (also referred to as a suicide gene) is a gene product that leads to the death of the cells during treatment with an agent that does not affect cells not expressing the negative selection marker, but is converted into a toxic agent by the negative selection marker.

Typically a positive selection marker is Puromycin (or other antibiotic) resistance gene, where the cells not expressing the resistance gene die on exposure of the toxic agent, and the negative selection marker is thymidine kinase converting e.g. gancyclovir or FiAU, to a toxic phosphorylated version thereof, causing death of cells expressing thymidine kinase.

The methods of the present invention typically use a pooled library of agents that can direct the activity of the genome perturbing agent to different parts of the genome, such as a pooled library of guide RNAs in a lentiviral vector. Other vector systems such as retroviral vectors or adenovirus assisted vectors are equally suitable. Upon transduction, the guide RNA is incorporated in the genome of the host cell. A gene that has been knocked out, interfered with, or activated can be identified by determining the incorporated guide RNA is such cells.

The screening method is based on a reporter gene or at least its regulatory elements with a positive and negative selection marker in a way that increased expression of the reporter gene leads to increased expression of the positive/negative selection markers, such that the expression of the makers genes is correlated with the expression of the reporter gene. The positive and negative selection marker is generally expressed an individual proteins and not as fusion proteins.

A positive selection marker allows the survival of the cells during treatment with an agent that kills cells not expressing the positive selection marker. A negative selection marker (“suicide gene”) leads to the death of the cells during treatment with an agent that does not affect cells not expressing the negative selection marker.

The methods of the invention include tagging the reporter gene with a fusion protein of puromycin resistance and thymidine kinase (Puro-TK cassette), and the genome perturbing agent is CRISPR/Cas9 driven by an inducible promoter for Cas9, directed by a pooled lentiviral library of gRNAs, and the survival of cells upon treatment with puromycin and gancyclovir/FiAU, with and without triggering of the pathway. The methods steps are the same for other combinations of a positive selection and a negative selection marker, genome perturbing agent, and pooled library.

Applying the trigger of a given pathway to the cells leads to the increased expression of the reporter gene of that pathway, leading to expression of the positive/negative selection marker protein (Puro-TK), as well. Triggering the pathway and thereby increasing the expression of the Puro-TK protein leads to survival of these cells during puromycin treatment, and death of the cells during treatment with FiAU/ganciclovir (or any other pro-drug that are converted to toxic compounds by thymidine kinase, but otherwise have minimal effect on cells not expressing thymidine kinase).

In the methods of the present invention pilot experiments are generally performed to optimise the expression of the reporter cassette and to determine optimal antibiotic concentrations for selecting cells of interest based on the viability of these cells.

Depending on the reporter gene, its activity may be absent upon control conditions in the absence of a trigger.

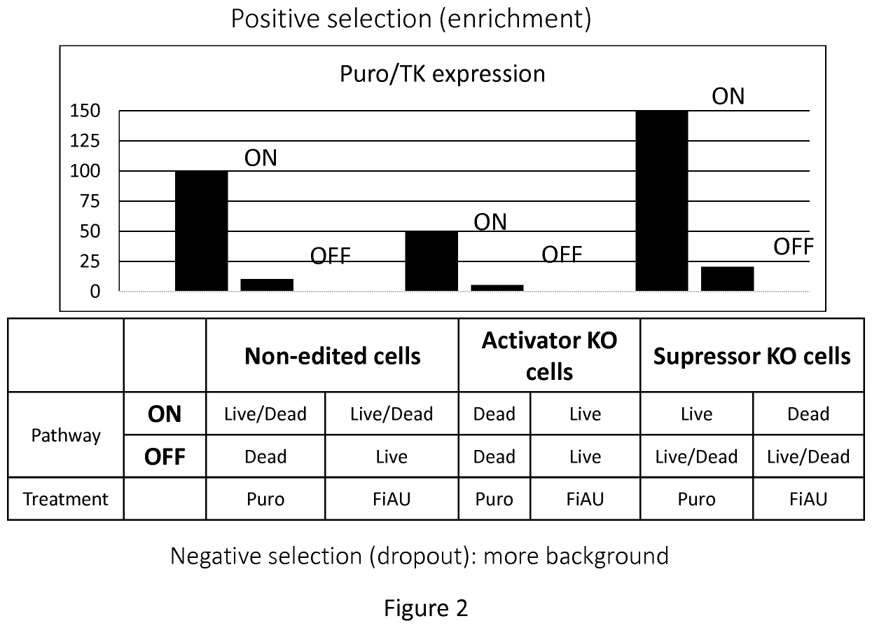

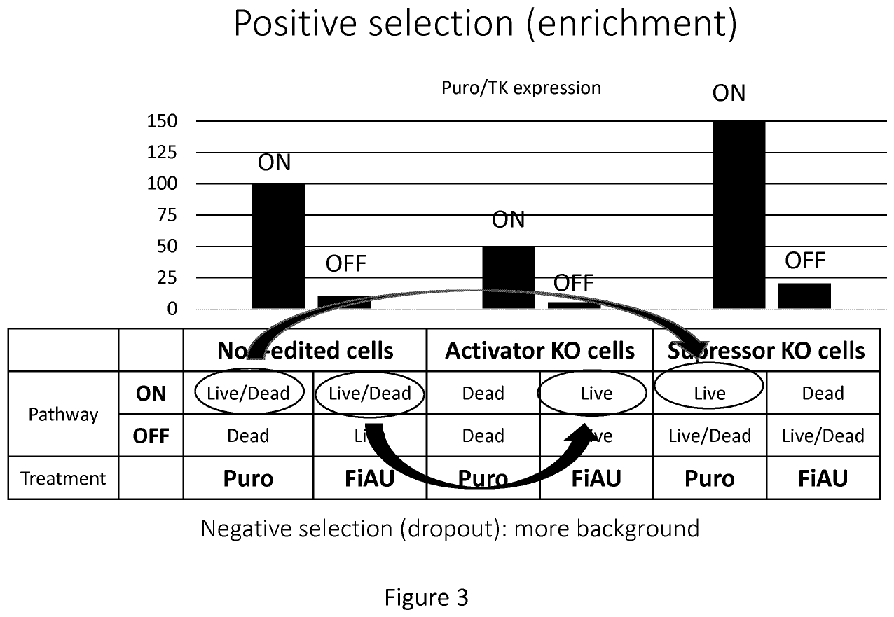

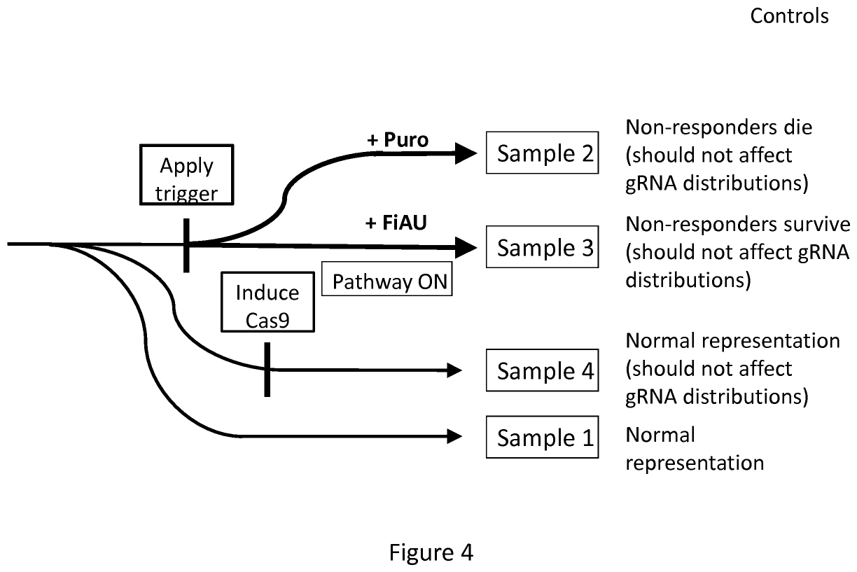

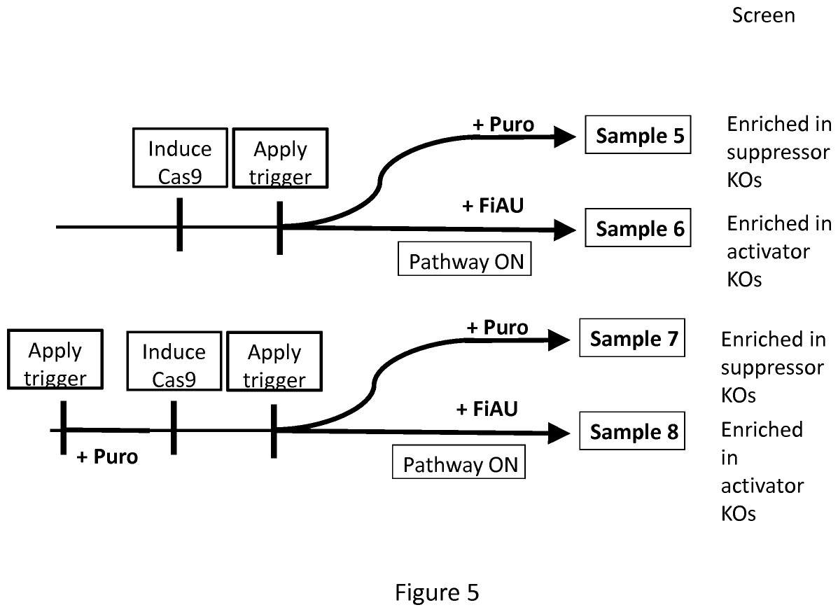

In order to be able to screen for both activators and suppressors of the pathway, the pathway is activated in control condition to such an extent that a decrease as well an increase of reporter gene activity can be provoked with a measurable read out on cell viability. This level of triggering is also referred to as “the middle of the dynamic range”. This allows to measure as well the effect of inactivated activator genes as of de-activated suppressor genes. This concept is illustrated in the FIGS. 2 and 3, wherein the control condition shows an arbitrary level of “100”.

It is also possible to increase the trigger to induce the reporter-2A-Puro/TK expression to maximal (saturation) level, which would favour discovery of activators of the pathway, or to decrease the trigger applied to induce the reporter-2A-Puro/TK to a low level, which would favour discovery of suppressors of the pathway.

To optimise the read out of the methods of the invention puromycin and FiAU is titrated to kill most of triggered cells, in order to decrease background by killing most of the cells that are not edited by Cas9, or where editing happens in genes that are not relevant to the pathway in question.

The invention as described above in the specification and below in the examples refers to embodiments wherein a reporter gene is chosen which is inactive in control conditions and wherein expression occurs upon activation of the pathway, leading to increased levels of Puro and Thymidine kinase, leading to respectively increased resistance to puromycin and decreased resistance to FiAU.

In alternative embodiments the method is used in situations wherein the reporter gene of a pathway is active under control conditions. Activation of such reporter gene leads to decreased resistance to puromycin and increased resistance to FiAU.

### EXAMPLES

### Example 1

### Screening for Activators/Suppressors

The general outline of the methods of the invention are outlined in FIGS. 4 and 5 and explained in more detail below.

A cell line is transfected with an inducible Cas9 construct

The cell lines is transduced with lentiviral gRNA library. In the case of a lentiviral library, the gRNAs are integrated in the genome of the host cells.

The obtained cell population is a mixture of cells containing no gRNA, containing a gRNA irrelevant for the pathway of interest and a small number of cells containing guide RNA which will inactivate an activator or a suppressor of the pathway of interest

The cell population is divided in the following samples: samples 1 to 4 are controls (Cas9 and activation of the pathway do not occur together)

Sample 1 (baseline) are untreated cells that represent the baseline distribution of gRNAs (the frequency of the gRNAs is similar). Cas9 is not induced, the cells behave as if there is no gRNA present.

Sample 2 (Puro control)is a cell population wherein Cas9 is not induced but wherein the pathway is activated. The activated cells are treated with puromycin. Puromycin treatment will kill the cells wherein the pathway is not activated , but this should not affect gRNA distribution. It may control for insertion events causing spontaneous activation of the pathway.

Sample 3 (FiAU control) is a cell population wherein Cas9 is not induced but wherein the pathway is activated. The activated cells are treated with FiAU. FiAU treatment will kill the cells that activated the pathway, and non-responders survive (should not affect gRNA distributions). It may control for insertion events causing spontaneous suppression of the pathway.

Sample 4 (Cas9 control) is a population of cells that were edited by Cas9, the pathway is not activated. Since Cas9 editing should not affect the gRNA distribution until the cells are subjected to selective pressure, this should be also normal representation.

Sample 5 to 8 represent experimental conditions wherein Cas9 is activated and the pathway is activated

Sample 5 (Suppressor KOs): the pathway is activated leading to expression of the Puro-TK genes. Cells are treated with puromycin to enrich for cells that express higher than average level of the positive selection marker. Cells that express higher level of the Puro marker will have a selective advantage, therefore the population remaining after the puromycin treatment is more likely to contain cells that are knocked out for suppressors of the pathway. A knockout of a suppressor leads to an increased activation of the pathway, resulting in increased puromycin expression, resulting in increased puromycin resistance.

Sample 6 (Activator KOs): The pathway is activated leading to expression of the Puro-TK genes. Cells are treated with FiAU to kill cells wherein the pathway is activated. Cells that express lower level of the TK marker will have a selective advantage, therefore this will enrich for cells that express lower than average level of the negative selection marker. This population is more likely to contain cells that are knocked out for activators of the pathway. A knockout of an activator leads to an decreased activation of the pathway, resulting in decreased TK expression, resulting in less conversion of FiAU into toxic compounds.

Sample 7: In an alternative strategy the trigger can be applied to limit the population of cells to those that successfully activate the pathway, before proceeding to [3] by treating the cells with puromycin before inducing Cas9. Indeed, the cells underwent a transfection with a Cas9 expression construct, a reporter—Puro/TK cassette and a transduction with a gRNA library.

Whether or not Cas9 is induced, the method relies on the reporter system of a pathway which can be triggered. When the pathway is triggered the puromycin resistance gene needs to be expressed rendering puromycin resistance. Cells which upon activation of the pathway do not survive puromycin treatment have an insufficient or defective reporter system and generate background in the experimental settings of the methods

Deep sequencing combined with bioinformatics is used to determine the frequency of the individual gRNAs in the different sample populations. This sequencing can be performed on the population of cells after completion of the experiment.

Overrepresented gRNAs already indicate which genes may be activator or suppressor genes. This can be performed by generating clonal population of the cells with assumed KO of activators or suppressors of the pathway.

### Example 2

### Detection of Activators/Suppressors of the ER-Stress Pathway

An inducible Cas9 as described in Cao et al. (2016) Nucleic Acids Res. 44, e149-e149, is knocked in to the AAVS1 locus of stem cells as in Ordovas et al (2015) Stem Cell Reports 5, 918-931.

As reporter gene for the ER-stress pathway, gene HSPAS/Grp78/BiP is selected as in (Wink et al. (2014) Chemical Res. Toxicol. 27, 338-355; Hiemstra et al. 2014), and a cassette containing Puro/TK markers is introduced in the stem cells essentially as described in (Roberts et al. (2017) Mol Biol Cell. 28, 2854-2874; Haupt et al. 2017). Alternatively, a gene construct containing only the regulatory elements operationally lined with expression of the Puro/TK markers as could be used (example of an NK-kB reporter is in (Ordovas, Boon et al. 2015)).

The constitutive expression of Puro and TK marker is determined and the survival rate of the cells upon treatment with different concentrations of puromycin and FiAU is determined.

Stem cells are transduced with a (preferably barcoded) lentiviral guideRNA library as described in (Shalem et al. (2014) Science 343, 84-87; Wang et al. (2014) Science 343, 80-84)

Stem cells are differentiated into stellate cells and subsequently cultivated as described in Sancho-Bru et al. (2011) Journal of Hepatology 54, 98-107.

After differentiation, the expression of the selection markers and the survival rate upon treatment puromycin and FiAU is determined to verify whether differentiation influence the expression level of the reporter gene.

Cas9 expression is induced using doxycycline as in (Cao, Wu et al. 2016).

Cells are treated with tunicamycin to activate the pathway.

Cell viability is determined as outlined above, and the identity of the gRNA is determined as described in the information of the supplier of the library.

1.-15. (canceled)

16. A method for pooled screening using RNA-guided nucleases for identifying genes in a pathway of interest, the method comprising:
providing a cell line with an CRISPR/Cas9 expression cassette;
transfecting the cell line with a reporter construct of a reporter gene in a pathway of interest, wherein the reporter construct comprises at least the regulatory elements of the reporter gene operably linked to a positive and negative selection marker, whereby a modified expression of the reporter gene leads to a modified expression of the positive and negative selection marker;
introducing in the cell line with the reporter construct a pooled library of CRISPR/Cas9 guide RNA;
cultivating the population of cells;
inducing Cas9 activity, thereby introducing a modification in the genome of cells which comprises a guide RNA;
activating the pathway of interest and contacting part of the cells with a positive selection agent and part of the cells with a negative selection agent,
wherein cells which survive treatment of the positive selection agent are indicative of being cells wherein a suppressor of the pathway is inactivated, and
wherein cells which survive treatment with the negative selection agent are indicative of being cells wherein an activator of the pathway is inactivated; and
identifying within the cells with increased viability, the guide RNA within the cells, where the gene that has been targeted is identified as a gene involved in the pathway.

17. The method according to claim 16, wherein the reporter gene is a gene which is constitutively expressed, allowing the measurement of a downregulation or an upregulation of the activity of the reporter gene, or
wherein, during control and experimental conditions, the reporter gene is activated to a certain level, allowing the measurement of a downregulation or a further upregulation of the activity of the reporter gene.

18. The method according to claim 16, wherein the CRISPR/Cas9 expression cassette is an inducible CRISPR/Cas9 expression cassette.

19. The method according to claim 16,
whereby an increased expression of the reporter gene leads to an increased expression of the positive and negative selection marker, or
whereby a decreased expression of the reporter gene leads to a decreased expression of the positive and negative selection marker.

20. The method according to claim 16, wherein the reporter construct comprises the regulatory elements of the reporter gene and the coding sequence of the reporter gene, and wherein the reporter gene is operably linked to a positive and negative selection marker.

21. The method according to claim 16, wherein the positive marker is a gene which expression converts a toxic positive selection agent into a non-toxic compound.

22. The method according to claim 16, wherein the positive marker is puromycin N-acetyl-transferase (PURO) and the positive selection agent is puromycin.

23. The method according to claim 16, wherein the negative marker is a gene which expression converts a non-toxic negative selection agent into a toxic compound.

24. The method according to claim 23, wherein the negative marker is thymidine kinase and the negative selection agent is FiAU.

25. The method according to claim 16, wherein the cells transfected with the expression cassette are stem cells.

26. The method according to claim 25, wherein prior to the induction of Cas9 the stem cells are differentiated into a desired cell type.

27. The method according to claim 25, wherein the stem cells are iPSC cells.

28. The method according to claim 25, wherein the stem cells are differentiated into stellate cells.

29. The method according to claim 16, wherein the RNA library is a barcoded library and wherein the identity of the gene involved in the pathway of interest is identified by the barcode corresponding to the guide RNA.

30. The method according to claim 16, further comprising determining the expression of the protein, the level of mRNA, or determining the genomic sequence of the gene.
